(19) United States
(12) Patent Application Publication
(10) Pub. No.: US 2005/0080434 A1
(43) Pub. Date: Apr. 14, 2005

(54) Laparoscopic retractable dissector and suture and needle passer

(75) Inventors: Steve Y. Chung, Los Angeles, CA (US); Gerhard J. Fuchs, Los Angeles, CA (US)

Correspondence Address: DAVIS WRIGHT TREMAINE LLP, 865 FIGUEROA STREET, SUITE 2400, LOS ANGELES, CA, 90017-2566, US

(73) Assignee: Cedars-Sinai Medical Center, Los Angeles, CA (US)

(21) Appl. No.: 10/842,737

(22) Filed: May 10, 2004

Related U.S. Application Data
(60) Provisional application No. 60/509,745, filed on Oct. 8, 2003.

(51) Int. Cl. A61B 17/10; A61B 17/12

(52) U.S. Cl. 606/148; 606/144; 606/139

(57) ABSTRACT

Described herein is a device that may be used in various applications, such as medical applications, and, particularly in laparoscopic procedures, to manipulate and/or introduce objects within or into the body. In one embodiment of the invention, a device includes a cannula with a handle that remains exterior to the body and an extendable member on a portion of the device that may be inserted into the body. The handle, which controls the extendable member, may be moved in an axial direction to extend or retract the extendable member from the interior of the cannula. It may also be rotated to swivel the extendable member. Furthermore, the extendable member may include at least one hole that may be used to, for example, grasp a surgical suture or suture needle. Thus, the device may be used to introduce such objects into the body and manipulate them therein.

### CROSS REFERENCE TO RELATED APPLICATIONS

This application claims the benefit of U.S. provisional patent application Ser. No. 60/509,745, filed Oct. 8, 2003, which is incorporated by reference herein in its entirety.

### FIELD OF THE INVENTION

The invention relates to the field of medical devices. More particularly, the invention relates to medical devices that may be used in connection with laparoscopic medical procedures, such as those involving the introduction, manipulation and/or tying of medical sutures.

### BACKGROUND OF THE INVENTION

Laparoscopic techniques are commonly used to perform a variety of surgical procedures. In general, laparoscopic techniques involve at least one small incision or puncture through the abdomen or other region of the body. Various devices can be inserted through the at least one incision or puncture wound to gain access to the interior regions of the body. An astounding array of surgical procedures may then be performed, including, for example, various abdominal procedures (e.g., gastric bypass, hernia repair, pyeloplasty), organ removal (e.g., appendectomy, nephrectomy, adrenalectomy, hysterectomy), endoscopic procedures, vein harvesting, lumbar fusion and others.

One significant challenge to the successful implementation of laparoscopic procedures is the difficulty associated with manipulating various objects inside the body. These objects include tissues, organs, tumors, blood and lymphatic vessels and other components of internal anatomy, as well as the foreign objects that may be introduced and used in the course of a surgical procedure, such as surgical sutures, suture needles, surgical sponges and the like. To that end, a host of medical devices have been developed and are commercially available to assist surgeons in performing operations. By way of example, the ENDO MINI-RETRACT (available from United States Surgical Corporation; Norwalk, Conn.) may be used in connection with various laparoscopic techniques. However, while this particular device may be used to manipulate objects inside the body, its use is limited by its linear, non-swiveling range of motion. It also cannot be used to manipulate surgical sutures or suture needles.

There is a need in the art for a device that may be used in connection with laparoscopic techniques. There is a further need in the art for a device that may be used in connection with such techniques to manipulate surgical sutures and suture needles in the body. There is a still further need in the art for a device that may be used in connection with such techniques to introduce surgical sutures into the body, and to thereafter manipulate them.

### SUMMARY OF THE INVENTION

The invention is based on a device that may be used in connection with various laparoscopic procedures, and methods of using the same. For instance, the device is particularly well suited to introducing and/or manipulating surgical sutures and/or suture needles into and within the body. The device may be used to manipulate objects inside the body, such as, but not limited to, blood and lymphatic vessels and tissue, as well as other non-biological objects, such as surgical sponges. The device of the invention can also be used to bluntly dissect tissue, to isolate large vessels, to permit the passage of suture ties intracorporally or extracorporally for vessel ligation, and to permit the passage of suture needles intracorporally or extracorporally for vessel ligation and oversewing.

To achieve the foregoing and in accordance with the purposes of the present invention, one embodiment of the present invention comprises a swivel or rotational feature, and a hole at the distal end of an extendable member. These features provide enhanced manipulation capability in vivo, and further enable one to introduce and manipulate surgical sutures during a laparoscopic procedure.

The device of the invention may include a cannula with a proximal end and a distal end. The proximal end may remain outside the body during a laparoscopic procedure, while the distal end may be inserted into the interior region of the body. The proximal end of the device may include a handle that is operated to control an extendable member at the distal end of the device. Axial motion of the handle with respect to the cannula results in a corresponding axial motion of the extendable member. Furthermore, rotational or circumferential motion of the handle results in similar movement of the extendable member. A locking mechanism enables the device user to lock the handle and extendable member in any desirable rotational orientation. The extendable member maintains a curvature when it is extended from the interior of the cannula, while also being capable of becoming substantially linear when it is retracted into the interior of the cannula. The extendable member may further include at least one hole. In particular embodiments, a hole may be included in the extendable member that is capable of accommodating a surgical suture and/or a suture needle.

In one aspect, the present invention is directed to a method for introducing a surgical suture into a body during a laparoscopic procedure. In another aspect, the present invention is directed to a method for manipulating a surgical suture. The device of the present invention can be used to introduce a surgical suture into a body and/or manipulate a surgical suture and suture needle in vivo. The surgical suture can be manipulated by movement of the device and/or user axial movement and/or rotation of the handle. By moving the device about within the body and, additionally, by extending and/or rotating the extendable member by user operation of the handle, the surgical suture may be deposited at a desirable location within the body (e.g., it may be passed behind a vessel and/or tied).

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

FIG. 1 is a superior view of the device of the invention in a retracted position in accordance with an embodiment of the present invention.

FIG. 2 is a posterior view of the device depicted in FIG. 1 in a retracted position in accordance with an embodiment of the present invention.

FIG. 3 is a lateral view of the device depicted in FIG. 1 in a retracted position in accordance with an embodiment of the present invention, wherein the handle of the device is shown rotated to an alternate position.

FIG. 4 is a superior view of the device depicted in FIG. 1 in an unretracted position in accordance with an embodiment of the present invention.

FIG. 5 is a lateral view of the device depicted in FIG. 4 in an unretracted position in accordance with an embodiment of the present invention.

FIG. 6 is a superior-lateral view of the device depicted in FIG. 4 in an unretracted position in accordance with an embodiment of the present invention, wherein the handle of the device is shown rotated to an alternate position.

FIG. 7 illustrates a user positioning the device of the invention to an unretracted position by axial movement of the handle towards the distal end of the device in accordance with an embodiment of the present invention.

FIG. 8 illustrates a user positioning the device depicted in FIG. 7 to a retracted position by axial movement of the handle towards the proximal end of the device in accordance with an embodiment of the present invention.

FIG. 9 illustrates a user rotating the handle of the device depicted in FIG. 8 in a retracted position in accordance with an embodiment of the present invention.

FIG. 10 is a perspective view of the handle and base member of the device of the invention in a retracted position in accordance with an embodiment of the present invention.

FIG. 11 is a perspective view of the extendable member of the device of the invention in an unretracted position in accordance with an embodiment of the present invention.

### DETAILED DESCRIPTION OF THE INVENTION

The invention is based on a device that may be used in connection with various laparoscopic procedures, and methods of using the same. The device itself is not limited to use in this particular field of art, as it may find application in other areas; particularly in a surgical setting, although use of the device and methods of the invention are not in any way limited to the medical field. With respect to laparoscopic procedures, however, the device is particularly well suited to introducing and/or manipulating surgical sutures and/or suture needles into and within the body. Other uses in the field of laparoscopy exist, as well. For example, the device may be used to manipulate objects inside the body, such as, but not limited to, blood and lymphatic vessels and tissue, as well as other non-biological objects, such as surgical sponges. The device can be also used to bluntly dissect tissue, to isolate large vessels, to permit the passage of suture ties intracorporally or extracorporally for vessel ligation, and to permit the passage of suture needles intracorporally or extracorporally for vessel ligation and oversewing.

The device of the invention may be similar in configuration to the ENDO MINI-RETRACT device available from United States Surgical Corporation. However, it possesses distinct advantages over this device owing to several novel features being included therein. These advantages allow one to use the device of the present invention to introduce and manipulate surgical sutures and suture needles, while the ENDO MINI-RETRACT device cannot be used in this fashion.

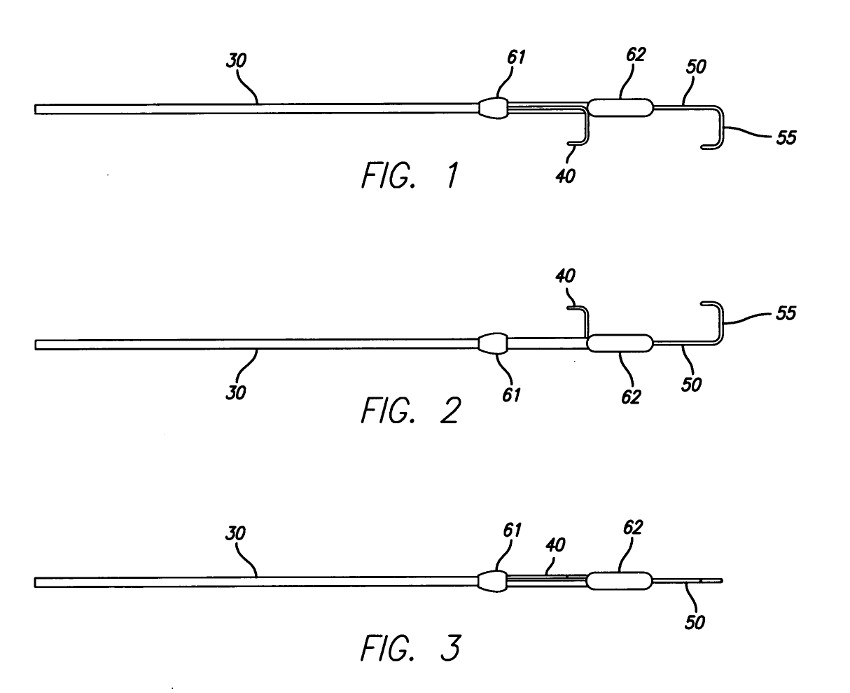

Referring to FIGS. 1-3, in one embodiment, the device of the invention may include a cannula 30 with a proximal end and a distal end. The proximal end may remain outside the body during a laparoscopic procedure, while the distal end may be inserted into the interior region of the body. References herein to distal and proximal ends shall be with reference to this overall configuration of the system. Thus, individual mechanical elements included in the device of the invention may be described with reference to their individual proximal and distal ends, which shall have a similar orientation as that described above with respect to the cannula.

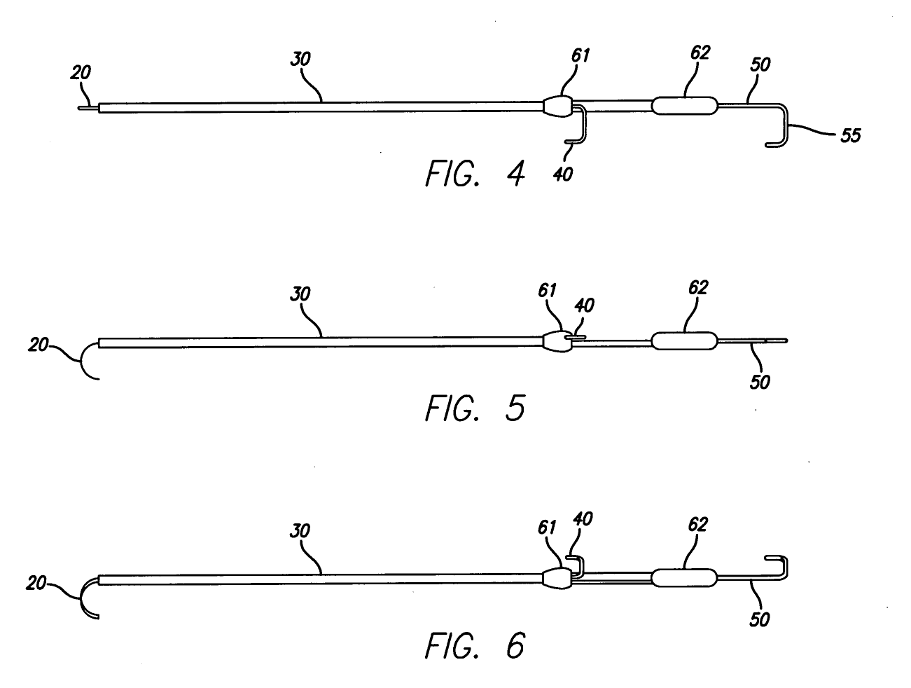

As illustrated in FIGS. 4-6, the proximal end of the device may include a handle 40 that is operated to control an extendable member 20 at the distal end of the device. The handle 40 may be of any suitable configuration. In various embodiments, it may be contoured or otherwise shaped to accommodate a user's fingers. To that end, the handle may include contours, finger rests, non-slip surfaces and other elements that may provide enhanced stability, control and ease of operation, as will be readily appreciated by one of skill in the art. FIG. 10 depicts a close-up view of the handle 40 and illustrates an embodiment of the invention wherein the handle contour 45 is a J-shaped curve. In other embodiments, the handle contour 40 may take various shapes, as for example, a C-shaped curve or an arc wherein the radius and extent of the arc may suitably be changed depending on the surgical procedure to be performed and the needs of the user.

As seen in FIGS. 1-6, a base member 50 may be included in the device and mechanically affixed to the cannula 30 such that it is not moveable with respect thereto. This base member 50 may provide additional stability for a user in manipulating the handle, in both axial and circumferential directions. In an embodiment of the invention, the base member 50 may also include contours, finger rests, non-slip surfaces and other elements that may provide enhanced stability, control and ease of operation, as will be readily appreciated by one of skill in the art. FIG. 10 depicts a close-up view of the base member 50 and illustrates an embodiment of the invention wherein the base member contour 55 of the base member 50 is a J-shaped curve. In other embodiments, the contour of the base member 50 may take various shapes, as for example, a C-shaped curve or an arc wherein the radius and extent of the arc may suitably be changed depending on the surgical procedure to be performed and the needs of the user.

As illustrated in FIGS. 1-6, the device of the invention may also include circumferential projections 61 and 62 located between the cannula 30 and handle 40, and between the handle 40 and base member 50, respectively. It should be appreciated that there may be one or more circumferential projections and that they may be located at various locations within the device. Such circumferential projections may be composed of a variety of materials such as stainless steel and/or non-slip surfaces which would provide comfort and permit ease of use for the user. Circumferential projections 61 and 62 may function as finger rests, may provide a tactile indication for the user as to the positioning and degree of penetration of the device within a body, and may provide enhanced support, stability, control and ease of operation, as will be readily appreciated by one of skill in the art.

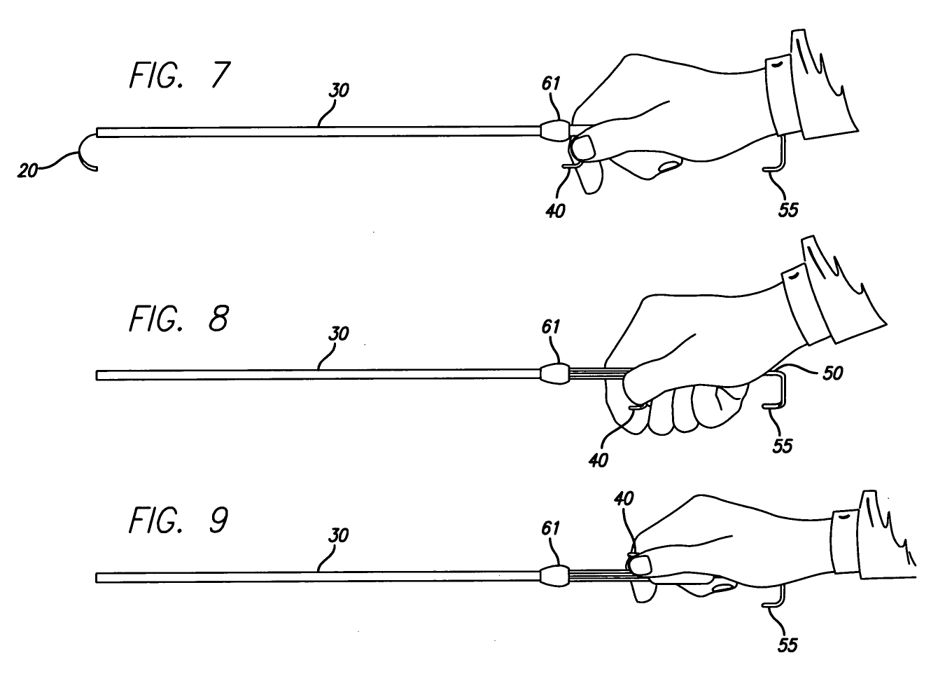

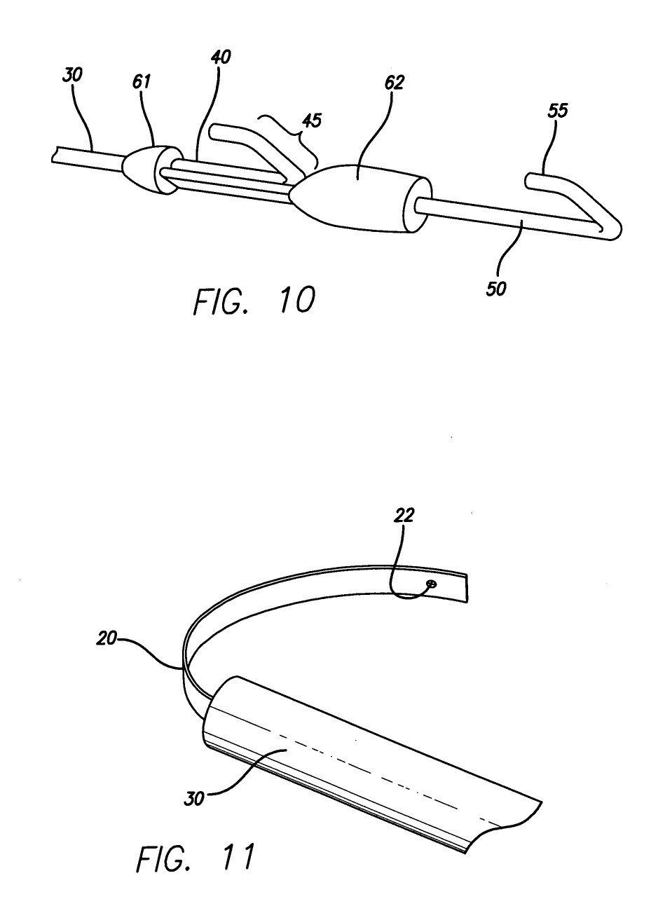

In an embodiment of the invention, the handle 40 may be mechanically connected to the extendable member 20 by way of an intermediate shaft that is slidably disposed within the cannula 30, or, in an alternate embodiment, the handle 40 itself may include a substantially elonged portion that is slidably disposed within the cannula 30 and mechanically connected to the extendable member 20. In yet a further alternate embodiment, the handle 40 and extendable member 20 may both be components of a unitary element. In each of these configurations, axial motion of the handle 40 with respect to the cannula 30 results in a corresponding axial motion of the extendable member 20. More specifically, as depicted in FIGS. 1-3 and 8, when the handle 40 is moved in the proximal direction with respect to the cannula 30, the extendable member 20 is at least partially retracted within the interior of the cannula 30. Conversely, as depicted in FIGS. 4-6, 7 and 11, when the handle 40 is moved in the distal direction with respect to the cannula 30, the extendable member 20 is extended at least partially outward from the interior of the cannula 30. Furthermore, as seen in FIGS. 3, 6 and 9, rotational or circumferential motion of the handle 40 results in similar movement of the extendable member 20, regardless of the degree to which the extendable member 20 is extended from the interior of the cannula 30. This swivel or rotational feature of the extendable member 20 can be achieved with one hand by rotating the handle 40 without changing the orientation of the entire instrument. A locking mechanism (not shown) may also be included; thereby enabling the device user to lock the handle 40 and extendable member 20 in any desirable rotational orientation with respect to the cannula 30. These are features of the device that are not incorporated in the ENDO MINI-RETRACT device, and which represent significant improvements. The ability to control not only axial, but also rotational motion of the extendable member 20 provides a greater range of motion that can be readily controlled by the device user. Moreover, it enables a user of the device to better control the device in various applications, such as in the introduction and/or manipulation of a surgical suture or suture needle. The further ability to lock the handle 40 and extendable member 20 in a specific rotational orientation with a locking mechanism is a still further improvement over the prior art device.

The extendable member 20 may be constructed of a metallic or other material that is malleable in nature. In one embodiment, the extendable member is constructed of a nickel cadmium composite material; although other materials can be used for this element, as will be readily appreciate by those of skill in the art. The material selected for the extendable member may be based upon its ability to maintain a curvature when it is extended from the interior of the cannula, while also being capable of becoming substantially linear when it is retracted into the interior of the cannula. Preferably, the material will not break or substantially fracture when subjected to these geometric alterations, particularly because such mechanical failure may result in damage to tissue and other anatomical structures in vivo.

FIG. 11 depicts an embodiment of the invention wherein the extendable member 20 assumes a C-shaped curvature when it is extended from the interior of the cannula 30 (i.e., the device of the invention is in an unretracted position). In alternate embodiments, the curvature of the extendable member 20 may assume various shapes when the device is in an unretracted position, as for example, a J-shaped curve or any arc shape wherein the radius and extent of the arc may suitably vary depending on the surgical procedure to be performed and the needs of the user. Furthermore, in another embodiment, the distal end of the extendable member 20 may include a blunt tip to prevent inadvertent penetration of tissue and other anatomical structures in vivo.

As illustrated in FIG. 11, the extendable member 20 may further include at least one hole 22. This hole 22 may be configured to any desirable size and shape. For instance, it may be roughly round, ovular, square or triangular, or, in alternate embodiments, it may be configured as an elongate slot either along the length or the width of the extendable member. Still further configurations of the hole or holes may be utilized, and, in embodiments wherein multiple holes are included, it will be readily appreciated by one of skill in the art that the holes need not be of the same size or shape. In particular embodiments, a hole may be included in the extendable member 20 that is capable of accommodating a surgical suture and/or a suture needle, as will be more readily apparent with reference to the Examples below, which describe various methods of using the device of the invention. In embodiments wherein the device of the invention is configured to accommodate suture and/or suture needle, the hole 22 may further include a notch which allows the extendable member 20 to better grip and capture the suture when the device is in a retracted position; thereby preventing slippage of the suture.

Moreover, the incorporation of at least one hole 22 into the extendable member 20 of the device is yet another improvement over the ENDO MINI-RETRACT device described above. This feature of the invention is particularly advantageous for embodiments of the device that are used in connection with the introduction and/or manipulation of surgical sutures and suture needles. In fact, a surgical needle and/or suture may be inserted through the at least one hole 22 in the extendable member 20.

In one embodiment, the device is constructed of stainless steel or other suitable material. However, in alternate embodiments, the device can be manufactured for one-time disposable use. Additionally, it should be appreciated that the device can have various dimensions depending on the surgical procedure to be performed and the needs of the user. For example, in one embodiment, the device of the present invention has a total length of about 50 cm in its retracted position, an outer sheath length of about 35 cm, a handle length of about 15 cm, a sheath width of about 7 mm, an extendable member length of about 5 cm, and an extendable member width of about 3 mm.

### EXAMPLES

The Examples described herein demonstrate various uses for the device of the present invention in connection with the introduction and manipulation of surgical sutures and suture needles. Such procedures may be particularly advantageous in the context of a laparoscopic procedure. However, as noted above, the device of the present invention has many uses beyond those illustrated herein, and the ensuing Examples are in no way intended to delineate the extent to which the device of the present invention may find application with a medical, or indeed any type of procedure.

### Example 1

### Introduction of a Surgical Suture

A device, as described above, is provided. The device includes a single hole 22 in its extendable member 20. A surgical suture is also provided. The extendable member 20 is extended from the interior of the cannula 30 of the device ex vivo by user operation of the handle 40 (i.e., the user moves the handle 40 axially towards the distal end of the device), and the surgical suture is inserted through the hole 22 in the extendable member 20. The extendable member 20 is then retracted into the interior of the cannula 30, again, by user operation of the handle (i.e., the user moves the handle 40 axially towards the proximal end of the device); thereby “grasping” the surgical suture.

The distal end of the device is then inserted into a body in the course of a laparoscopic procedure along with a length of the “grasped” surgical suture. Once inserted into the body, the extendable member 20 is extended by user movement of the handle 40 towards the distal end of the device. Optionally, the surgical suture is thereafter further manipulated by user rotation of the handle 40. By moving the device about within the body, by extending and/or rotating the extendable member 20 by user operation of the handle 40, and/or by locking the extendable member 20 and handle 40 with the locking mechanism in a specific rotational orientation at various points throughout the course of the laparoscopic procedure, the surgical suture may be deposited at a desirable location within the body (e.g., it may be passed behind a vessel and/or tied).

### Example 2

### In Vivo Manipulation of a Surgical Suture and Suture Needle

A device, as described above, is provided. The device includes a single hole 22 in its extendable member 20. A surgical suture connected to a suture needle is also provided. The surgical suture and suture needle are introduced into a body in the course of a laparoscopic procedure by a methodology conventionally used in the art. The distal end of the device is also introduced into the body.

The extendable member 20 is extended from the interior of the cannula 30 of the device in vivo by user operation of the handle 40 (i.e., the user moves the handle 40 axially towards the distal end of the device). The suture needle is passed entirely through the hole 22 in the extendable member 20, such that a portion of surgical suture remains in the hole 22. The suture needle is then disconnected (e.g., cut) from the surgical suture. The extendable member 20 is then retracted into the interior of the cannula 30, again, by user operation of the handle (i.e., the user moves the handle 40 axially towards the proximal end of the device); thereby “grasping” the surgical suture.

The surgical suture is thereafter manipulated by movement of the device and/or user axial movement and/or rotation of the handle 40. By moving the device about within the body, by extending and/or rotating the extendable member 20 by user operation of the handle 40, and/or by locking the extendable member 20 and handle 40 with the locking mechanism in a specific rotational orientation at various points throughout the course of the laparoscopic procedure, the surgical suture may be deposited at a desirable location within the body (e.g., it may be passed behind a vessel and/or tied).

While the description above refers to particular embodiments of the present invention, it should be readily apparent to people of ordinary skill in the art that a number of modifications may be made without departing from the spirit thereof. The accompanying claims are intended to cover such modifications as would fall within the true spirit and scope of the invention. The presently disclosed embodiments are, therefore, to be considered in all respects as illustrative and not restrictive, the scope of the invention being indicated by the appended claims rather than the foregoing description. All changes that come within the meaning of and range of equivalency of the claims are intended to be embraced therein.

1. A device, comprising:
a cannula;
a handle slidably disposed within said cannula; and
an extendable member in mechanical communication with said handle, such that axial and rotational movement of said handle with respect to said cannula results in corresponding axial and rotational movement of said extendable member, respectively, said extendable member having a curvature when extended from an interior of said cannula and having at least one hole disposed therein.

2. The device of claim 1, further comprising a base member mechanically affixed to said cannula.

3. The device of claim 1, further comprising an intermediate shaft mechanically connected at a distal end thereof to said extendable member and at a proximal end thereof to said handle, said intermediate shaft being slidably disposed within said cannula.

4. The device of claim 1, wherein said handle comprises a substantially elongated portion that is slidably disposed within said cannula and said handle is mechanically connected to said extendable member.

5. The device of claim 1, wherein said handle and said extendable member are components of a unitary element.

6. The device of claim 1, wherein said cannula and said handle are constructed of stainless steel.

7. The device of claim 1, where said cannula and handle are constructed of material suitable for disposable use.

8. The device of claim 1, wherein said extendable member is constructed of a malleable material.

9. The device of claim 1, wherein said extendable member is constructed of a nickel cadmium composite material.

10. The device of claim 1, wherein said hole further comprises a notch.

11. The device of claim 1, wherein a distal end of said extendable member is of a blunt shape.

12. The device of claim 1, further comprising at least one circumferential projection located between said cannula and a proximal end of said device.

13. The device of claim 1, further including a locking mechanism to lock said handle and said extendable member in a rotational orientation.

14. A device, comprising:
a cannula;
a handle slidably disposed within said cannula;
a base member mechanically affixed to said cannula; and
an extendable member in mechanical communication with said handle, such that axial and rotational movement of said handle with respect to said cannula results in corresponding axial and rotational movement of said extendable member, respectively, said extendable member having a curvature when extended from an interior of said cannula and having at least one hole disposed therein.

15. The device of claim 14, further including a locking mechanism to lock said handle and said extendable member in a rotational orientation.

16. A method for introducing a surgical suture into a body, comprising:
providing a device, comprising:
a cannula,
a handle slidably disposed within said cannula, and
an extendable member in mechanical communication with said handle, such that axial and rotational movement of said handle with respect to said cannula results in corresponding axial and rotational movement of said extendable member, respectively, said extendable member having a curvature when extended from an interior of said cannula and having at least one hole disposed therein;
extending said extendable member;
inserting said surgical suture through said hole;
retracting said extendable member; and
inserting a distal end of said device into said body to introduce said surgical suture therein.

17. The method of claim 16, wherein said device further comprises a locking mechanism to lock said handle and said extendable member in a rotational orientation, and wherein said method further comprises performing a locking action selected from the group consisting of locking said locking mechanism during the course of introducing said surgical suture into said body, unlocking said locking mechanism during the course of introducing said surgical suture into said body, and both locking and unlocking said locking mechanism during the course of introducing said surgical suture into said body.

18. A method for manipulating a surgical suture within a body, comprising:
inserting a device into said body, said device comprising:
a cannula,
a handle slidably disposed within said cannula, and
an extendable member in mechanical communication with said handle, such that axial and rotational movement of said handle with respect to said cannula results in corresponding axial and rotational movement of said extendable member, respectively, said extendable member having a curvature when extended from an interior of said cannula and having at least one hole disposed therein;
extending said extendable member;
inserting a suture needle attached to said surgical suture through said hole;
disconnecting said suture needle from said surgical suture;
retracting said extendable member; and
manipulating said surgical suture within said body with said device.

19. The method of claim 18, wherein said device further comprises a locking mechanism to lock said handle and said extendable member in a rotational orientation.

20. The method of claim 19, wherein manipulating said surgical suture within said body with said device further comprises performing a locking action selected from the group consisting of locking said locking mechanism while manipulating said surgical suture, unlocking said locking mechanism while manipulating said surgical suture, and both locking and unlocking said locking mechanism while manipulating said surgical suture.
